(19) United States
(12) Patent Application Publication
(10) Pub. No.: US 2021/0267704 A1
(43) Pub. Date: Sep. 2, 2021

(54) LIGHTING APPARATUS COVER

(71) Applicant: THE REGENTS OF THE UNIVERSITY OF CALIFORNIA, Oakland, CA (US)

(72) Inventors: James Todd Walker, La Jolla, CA (US); Kevin Cyrus Parvaresh, La Jolla, CA (US); Ian Michael Foran, La Jolla, CA (US)

(21) Appl. No.: 17/254,170

(22) PCT Filed: Jun. 17, 2019
(86) PCT No.: PCT/US2019/037530

§ 371 Date: Dec. 18, 2020

Related U.S. Application Data
(60) Provisional application No. 62/686,578, filed on Jun. 18, 2018.

(51) Int. Cl. A61B 46/10; A61B 90/30

(52) U.S. Cl. CPC A61B 46/10; A61B 90/30

(57) ABSTRACT

A apparatus to cover a lighting apparatus is provided. The apparatus includes a flexible material couplable to a lighting apparatus. A portion of the flexible material to cover a light-emitting surface of the lighting apparatus is transparent.

### CROSS REFERENCE TO RELATED APPLICATION

This application is a U.S. national phase of International Application No. PCT/US2019/037530, filed on Jun. 17, 2019, which claims priority to U.S. Provisional Application No. 62/686,578, filed Jun. 18, 2018, the contents of which are hereby incorporated herein by reference in their entirety.

### TECHNICAL FIELD

Various embodiments generally relate to a cover for lighting apparatuses. More particularly, various embodiments are related to a sterile protective cover for lighting apparatuses used within sterile environments.

### BRIEF SUMMARY OF THE EMBODIMENTS

Disclosed are systems and devices that relate to a lighting apparatus cover lower body negative pressure device. An apparatus to cover a lighting apparatus may include a flexible material couplable to a lighting apparatus. Coupling the flexible material to the lighting apparatus may cover a portion of the lighting apparatus with the flexible material. At least a portion of the flexible material may cover a light-emitting surface of the lighting apparatus may be transparent. Light emitted from the lighting apparatus may be able to pass through the flexible material onto an area of interest.

In embodiments, the lighting apparatus may be used in a surgical room.

In embodiments, the apparatus may be disposable.

In embodiments, the apparatus may further include a coupling mechanism to couple the flexible material to the lighting apparatus. The coupling mechanism may be coupled to a portion of the flexible material.

In embodiments, the coupling mechanism may include one or more of an adhesive, a strap, a tie, a plastic sealing feature, a popper, and a zip.

In embodiments, the apparatus may further include an opening to accept the lighting apparatus.

In embodiments, the opening comprises an elastic material to adapt to fit various sized lighting apparatuses.

Additional aspects of the present disclosure relate to an apparatus to cover a lighting apparatus. The apparatus may be to cover the lighting apparatus may include a flexible material to cover a portion of the lighting apparatus. The flexible material may be couplable to the lighting apparatus. The apparatus may include a rigid material to cover a light-emitting surface of the lighting apparatus. The rigid material may be bonded to the flexible material.

In embodiments, the lighting apparatus may be used in a surgical room.

In embodiments, the apparatus may be disposable.

In embodiments, the apparatus may further include a coupling mechanism to couple the flexible material to the lighting apparatus. The coupling mechanism may be integrated with the flexible material.

In embodiments, the coupling mechanism may include one or more of an adhesive, a strap, a tie, a plastic sealing feature, a popper, and a zip.

In embodiments, the apparatus may further include an opening to receive the lighting apparatus.

In embodiments, the opening may include an elastic material to adapt to fit various sized lighting apparatuses.

Additional aspects of the present disclosure relate to an apparatus to cover a lighting apparatus. The apparatus may include a flexible material couplable to a lighting apparatus. The apparatus may include an elastic opening in the flexible material to receive the lighting apparatus. The elastic opening may be adaptable to fit various sized lighting apparatuses The apparatus may include a rigid material to cover a light-emitting surface of the lighting apparatus.

In embodiments, the lighting apparatus may be used in a surgical room.

In embodiments, the apparatus may be disposable.

In embodiments, the apparatus may further include a coupling mechanism to couple the flexible material to the lighting apparatus.

In embodiments, the coupling mechanism may include one or more of an adhesive, a strap, a tie, a plastic sealing feature, a popper, and a zip.

In embodiments, the rigid material may direct light emitted from the light-emitting surface onto an area of interest.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 illustrates an example cover, in accordance with one embodiment of the technology described herein.

FIG. 2 illustrates a sealing mechanism of an example cover, in accordance with one embodiment of the present disclosure.

FIG. 3 illustrates an example sterile bag into which an example cover is placed, in accordance with one embodiment of the present disclosure.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION OF THE EMBODIMENTS

Surgical site infections (SSIs) are a significant cause of morbidity and mortality for all surgical subspecialties. Between 160,000-300,000 SSIs occur each year, accounting for two to six percent of all patients undergoing surgery. As a result, patient morbidity and healthcare costs are very high. Patients who are diagnosed with a SSI have a two to eleven times higher risk of death than patients without SSIs. SSIs can result in serious injury to tissues and organs, loss of a limb, increased hospital stays, and long-term disability. Additionally, SSIs may account for 3.5-10 billion dollars of health expenditures in the United States alone. Measures that decrease the rate of SSIs are therefore beneficial to patients, as well as the healthcare system as a whole.

Nationwide scrutiny of SSIs has improved hospital surveillance and practice guidelines corresponding to decreasing the risk of infections. In other words, there is an increasing priority for hospitals to implement protocols to decrease SSIs. One etiology of SSIs is the operating room, where the creation and maintenance of a sterile field during surgical procedures is directly correlated to reducing SSIs. As described above, SSIs are a serious medical problem, and extreme measures are taken to ensure low microbe counts within sensitive spaces, such as operating rooms.

Surgical lamps, and other lighting apparatuses used in sensitive spaces, have been designed to allow the operator to move the lighting apparatus into different positions or angles throughout a procedure. Lighting apparatuses may be positioned directly over the patient. Generally, while devices used in the operating room must either be disposed of and replaced or undergo a sterilization process prior to proceeding with another surgery, some devices are not fully sterilized.

For example, lighting apparatuses may be cleaned infrequently, particularly around the back of the lighting apparatuses, and may therefore accumulate microbes which could fall into vulnerable sites. In order to allow the operator to safely manipulate the lighting apparatus and maintain sterility, several disposable sterile lighting apparatus handle covers have been designed. Currently, sterile lighting apparatus handle covers are disposed for each surgical procedure, and subsequently new covers are used for each procedure. However, the remainder of the lighting apparatus is exposed to airborne debris and bacteria.

Airborne bacteria has proven to be a source of contamination during surgery. During the course of a procedure, lighting apparatuses are frequently manipulated and placed directly above vulnerable sites, such as wounds. Existing lighting apparatuses are not disposable and are often not sterile. They collect dust, particulate debris, blood, and tissue from patients during the course of various operations. These contaminants can fall from the lighting apparatus directly onto the vulnerable site, causing contamination, leading to, for example, patient post-operative wound infections.

Embodiments of the system and methods disclosed herein provide a cover to enclose lighting apparatuses and protect sterile environments. The cover may be sterile and semi-disposable. In embodiments, the cover may be a sterile protective cover for lighting apparatuses used within sterile environments, such as, for example, surgical operating rooms or procedure rooms. The sterile covers may enable the use of necessary, non-disposable equipment in sensitive spaces while reducing exposure of contaminants from the lighting apparatus to patients. In some embodiments, the cover may be a disposable, transparent, collapsible, and sterile covering that can cover an entire lighting apparatus, while allowing transmission of light rays through the material.

In some embodiments, the cover may be a disposable, transparent, sterile drape designed to maintain a sterile environment around the lighting apparatus. The cover may protect the sterile environment from the lighting apparatus, while still allowing adjustment of lighting apparatus position and angle (if the lighting apparatus allows for this) and allowing light rays to be directed onto the area of interest.

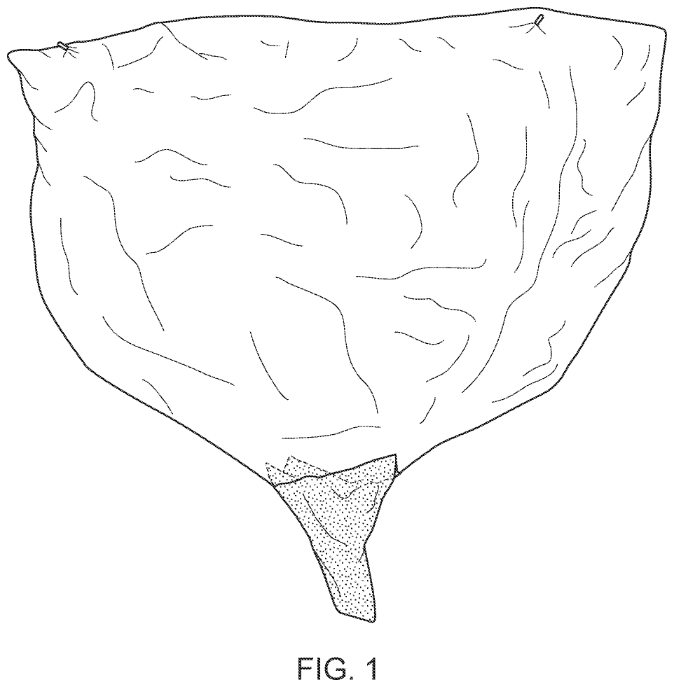

FIG. 1 illustrates an example cover in accordance with one embodiment of the technology described herein. As illustrated, the cover includes one or more sheets of a material that is flexible, transparent, and sterile. The material may include one or more of a PETG plate, a LDPE, HDPE, mylar, polycarbonate, vinyl, acrylic, and other materials. The covers may be manufactured either as one, or several sheets, of material that are formed to surround a lighting apparatus. The covers may include an opening to receive and surround the lighting apparatus and a sealing mechanism at one end of the cover to couple and secure the cover to the lighting apparatus.

In some embodiments, the cover may include a rigid, transparent material, such as, for example, a PETG plate, to cover the light-emitting surface of the lighting apparatus. The rigid material may prevent unwanted reflections and refractions of the light rays that may result from using a more flexible material. The rigid material may ensure that light rays are directed to an area of interest in an uninterrupted way. In some embodiments, the rigid material may filter the light, such that the light transmitted from the rigid material is diffuse, concentrated, or otherwise affected. The rigid material may include one or more openings to accommodate feature of the lighting apparatus, such as, for example, a handle or hinge. The one or more openings may include pockets of flexible material to provide maneuverability of the feature, while maintaining the sterile environment.

In embodiments, a flexible, transparent material, such as, for example, LDPE, may be coupled to the rigid material and conform around the rest of the lighting apparatus. The flexible material may be coupled to the rigid material by one or more of solvent bonding, UV bonding, ultrasonic welding, mechanical fastening, heat bonding, and other coupling mechanisms.

In embodiments, the cover may also include a sealing mechanism to secure the cover to the lighting apparatus, thereby protecting the sterile environment around the lighting apparatus. The sealing mechanism may be coupled to the body of the cover on a first side of the sealing mechanism. A second side of the sealing mechanism may be used to couple and secure the cover to the lighting apparatus. The second side may include an adhesive, Velcro, plastic sealing feature, popper, zip, or other sealing mechanisms. In embodiments, the second side may be couplable, or otherwise easily removable, from the lighting apparatus.

In some embodiments, the cover may include an opening to receive and surround multiple different lighting apparatuses. In some embodiments, a cover may fit around different sized and shaped lighting apparatuses. In other embodiments, multiple sizes of the covers may be designed such that a first size of covers fits a first set of lighting apparatuses within a first range of volume, and a second size of covers fits a second set of lighting apparatuses within a second range of volume, and so on. In embodiments, the cover may be custom designed to tightly wrap around each surface of a given lighting apparatus, such as the light-emitting surface, the handles, the back, and any other surfaces. After the covers are manufactured, the covers may be sterilized and stored in a sterile bag until use.

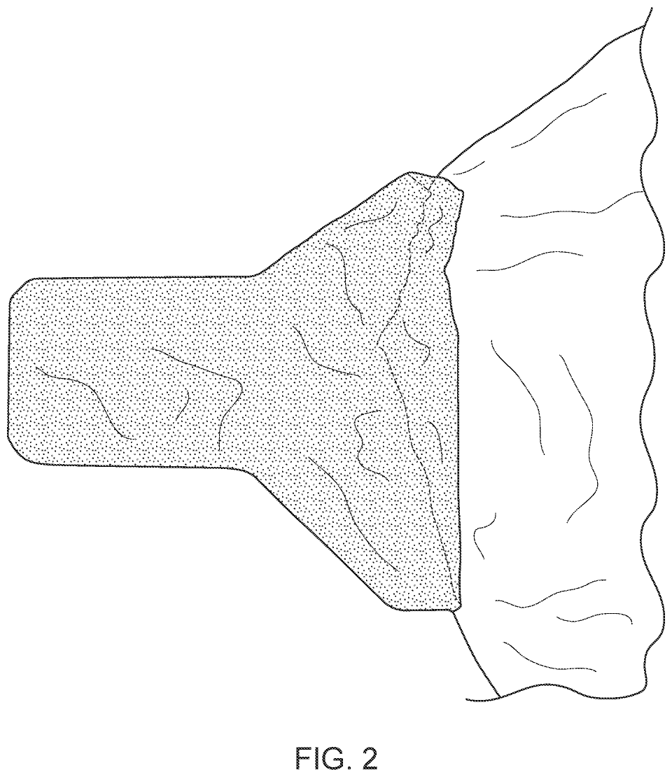

FIG. 2 illustrates an adhesive of an example cover, in accordance with one embodiment of the present disclosure. As illustrated, the sealing mechanism may be an adhesive strip coupled to an end of the cover. The adhesive strip may be couplable to a surface opposite the light-emitting surface of the lighting apparatus to create a sterile environment.

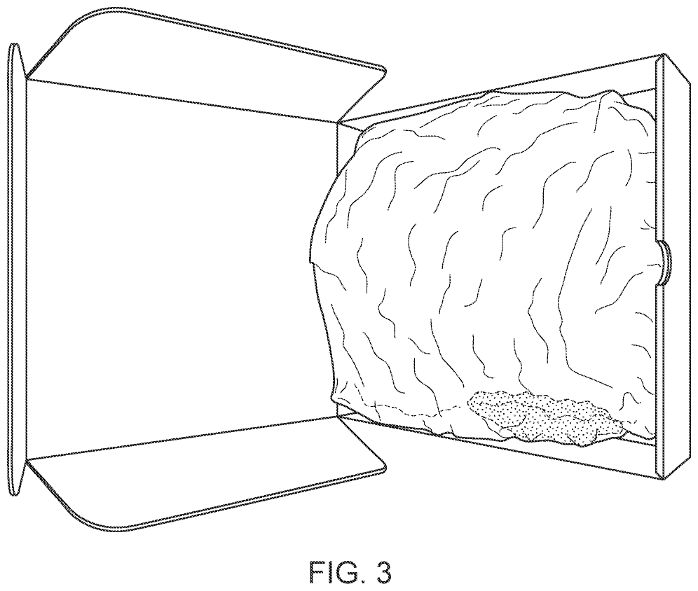

FIG. 3 illustrates an example sterile bag into which an example cover is placed, in accordance with one embodiment of the present disclosure. As illustrated, one or more covers may be packaged into a sterile bag to preserve the sterility of the covers while in transport and before use.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that can be included in the disclosed technology. The disclosed technology is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the technology disclosed herein. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. An apparatus comprising a flexible material couplable to a lighting apparatus, such that coupling the flexible material to the lighting apparatus covers a portion of the lighting apparatus with the flexible material, wherein at least a portion of the flexible material covering a light-emitting surface of the lighting apparatus is transparent, such that light emitted from the lighting apparatus is able to pass through the flexible material onto an area of interest.

2. The apparatus of claim 1, wherein the lighting apparatus is used in a surgical room.

3. The apparatus of claim 1, wherein the apparatus is disposable.

4. The apparatus of claim 1, further comprising a coupling mechanism to couple the flexible material to the lighting apparatus, wherein the coupling mechanism is coupled to a portion of the flexible material.

5. The apparatus of claim 4, wherein the coupling mechanism comprises one or more of an adhesive, a strap, a tie, a plastic sealing feature, a popper, and a zip.

6. The apparatus of claim 1, further comprising an opening to accept the lighting apparatus.

7. The apparatus of claim 6, wherein the opening comprises an elastic material to adapt to fit various sized lighting apparatuses.

8. An apparatus comprising:
a flexible material to cover a portion of the lighting apparatus, wherein the flexible material is couplable to the lighting apparatus;
a rigid material to cover a light-emitting surface of the lighting apparatus, wherein the rigid material is bonded to the flexible material.

9. The apparatus of claim 8, wherein the lighting apparatus is used in a surgical room.

10. The apparatus of claim 8, wherein the apparatus is disposable.

11. The apparatus of claim 8, further comprising a coupling mechanism to couple the flexible material to the lighting apparatus, wherein the coupling mechanism is integrated with the flexible material.

12. The apparatus of claim 11, wherein the coupling mechanism comprises one or more of an adhesive, a strap, a tie, a plastic sealing feature, a popper, and a zip.

13. The apparatus of claim 8, further comprising an opening to receive the lighting apparatus.

14. The apparatus of claim 13, wherein the opening comprises an elastic material to adapt to fit various sized lighting apparatuses.

15. An apparatus comprising:
a flexible material couplable to a lighting apparatus;
an elastic opening in the flexible material to receive the lighting apparatus, wherein the elastic opening is adaptable to fit various sized lighting apparatuses;
a rigid material to cover a light-emitting surface of the lighting apparatus.

16. The apparatus of claim 15, wherein the lighting apparatus is used in a surgical room.

17. The apparats of claim 15, wherein the apparatus is disposable.

18. The apparatus of claim 15, further comprising a coupling mechanism to couple the flexible material to the lighting apparatus.

19. The apparatus of claim 18, wherein the coupling mechanism comprises one or more of an adhesive, a strap, a tie, a plastic sealing feature, a popper, and a zip.

20. The apparatus of claim 15, wherein the rigid material directs light emitted from the light-emitting surface onto an area of interest.
